(19) United States
(12) Patent Application Publication
(10) Pub. No.: US 2007/0122841 A1
(43) Pub. Date: May 31, 2007

(54) Massively parallel synthesis of proteinaceous biomolecules

(76) Inventors: John J. Rajasekaran, Los Altos, CA (US); Gunjan Tiwari, Sunnyvale, CA (US); Edelmira Cabezas, Sunnyvale, CA (US); Jacqueline A. Fidanza, San Francisco, CA (US); Narayan Sundararajan, San Francisco, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/291,296

(22) Filed: Nov. 30, 2005

(51) Int. Cl. G01N 33/53; G01N 1/28; C12M 1/34

(52) U.S. Cl. 435/7.1; 435/287.2; 530/400; 427/2.11

(57) ABSTRACT

Methods for fabricating dense arrays of polymeric molecules in a highly multiplexed manner are provided using semiconductor-processing-derived lithographic methods. Advantageously, the methods are adaptable to the synthesis of a variety of polymeric compounds. For example, arrays of peptides and polymers joined by peptide bonds may be fabricated in a highly multiplexed manner.

### BACKGROUND OF THE INVENTION

1. Field of the Invention

The present invention relates generally to microarrays of bio-polymers, semiconductor lithographic technology, and synthetic organic chemistry.

2. Background Information

Microarrays of oligonucleotides, peptides, proteins, and or oligosaccharides continue to gain importance as powerful tools for research and diagnostic applications in the biomedical sciences. Oligonucleotide microarrays, for example, can be used to monitor gene expression and genetic mutations in a massively parallel manner. Proteinaceous microarrays provide the ability, for example, to characterize the molecular progression of disease, research cellular pathways, and perform high throughput screening in drug discovery applications. Peptide-containing arrays can serve as molecular probes for a variety of biological events, such as for example, the arrays can serve as antigens for antibody-antigen systems, ligands for cell receptor-ligand system, and substrates for enzyme-protein systems. The ability to collect large volumes of information is an integral part of biomarker discovery and personalization of medical treatments. Further, other applications in bioscience, such as for example, the analysis of the proteomic content of an organism, disease detection, pathogen detection, environmental protection, food safety, and biodefense are capable of benefiting from tools that allow rapid multiplexed interrogation of analyte samples.

As the genomic and proteomic knowledge base expands, so does the need for methods to collect, understand, and apply biologically relevant information. The drive towards personalized medicine magnifies these needs. Methods, such as analyses using microarrays that allow the use of small volumes of sample for highly multiplexed analysis, are valuable tools. Methods that provide for the controllable automated manufacture of arrays are similarly valuable.

### BRIEF DESCRIPTION OF THE FIGURES

FIGS. 1A through 1F demonstrate a method for the controllable synthesis of polymers on a solid support involving semiconductor lithography.

FIG. 2 provides chemical structure diagrams for exemplary molecules and functional groups.

FIG. 3 shows a method for derivatizing a SiO₂ surface and attaching a linker molecule to the derivatized surface.

FIG. 4 demonstrates a method for solid phase peptide synthesis according to embodiments of the invention.

FIG. 5 shows a photo generation of acid reaction and a resulting deprotection of an amino acid according to embodiments of the invention.

### DETAILED DESCRIPTION OF THE INVENTION

An array is an intentionally-created collection of molecules housed on a solid support in which the identity or source of a group of molecules is known based on its location on the array. The molecules housed on the array and within a feature of an array can be identical to or different from each other. Embodiments of the present invention provide methods for the synthesis of polymers on a solid support. Polymer synthesis according to embodiments of the invention can be accomplished in a manner that provides controlled-density microarrays comprised of peptides and or other small molecules. Embodiments of the present invention provide arrays of peptides of known sequence and controllable molecular density.

The features, regions, or sectors of an array may have any convenient shape, for example, circular, square, rectangular, elliptical, or wedge-shaped. In some embodiments, the region in which each distinct molecule is synthesized within a sector is smaller than about 1 mm², or less than 0.5 mm². In further embodiments the regions have an area less than about 10,000 μm² or less than 2.5 μm². Additionally, multiple copies of a polymer will typically be located within any region. The number of copies of a polymer can be in the thousands to the millions within a region. In general, an array can have any number of features, and the number of features contained in an array may be selected to address such considerations as, for example, experimental objectives, information-gathering objectives, and cost effectiveness. An array could be, for example, a 20×20 matrix having 400 regions, 64×32 matrix having 2,048 regions, or a 640×320 array having 204,800 regions. Advantageously, the present invention is not limited to a particular size or configuration for the array.

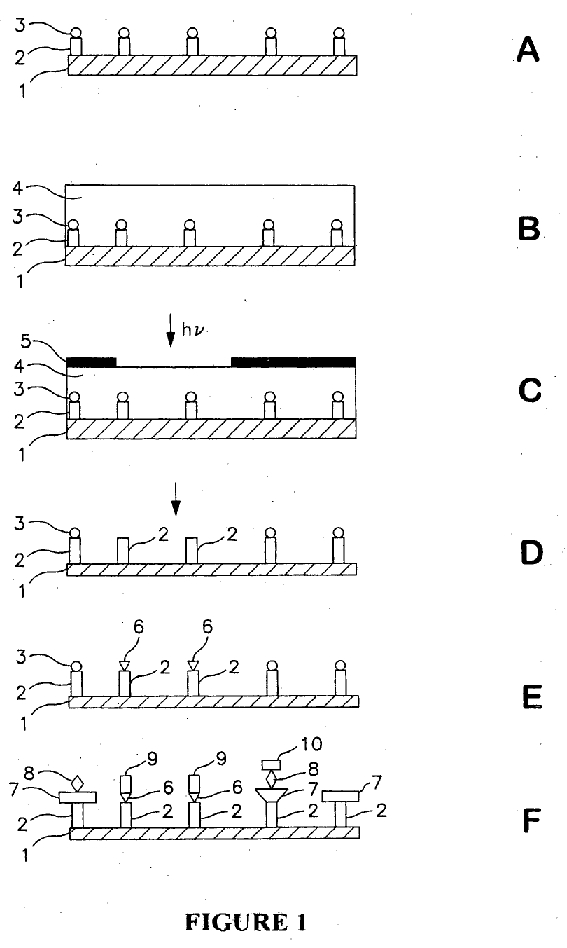

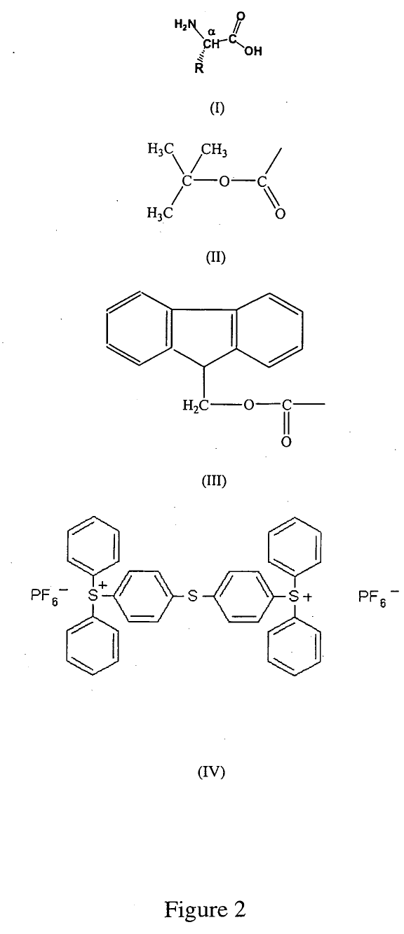

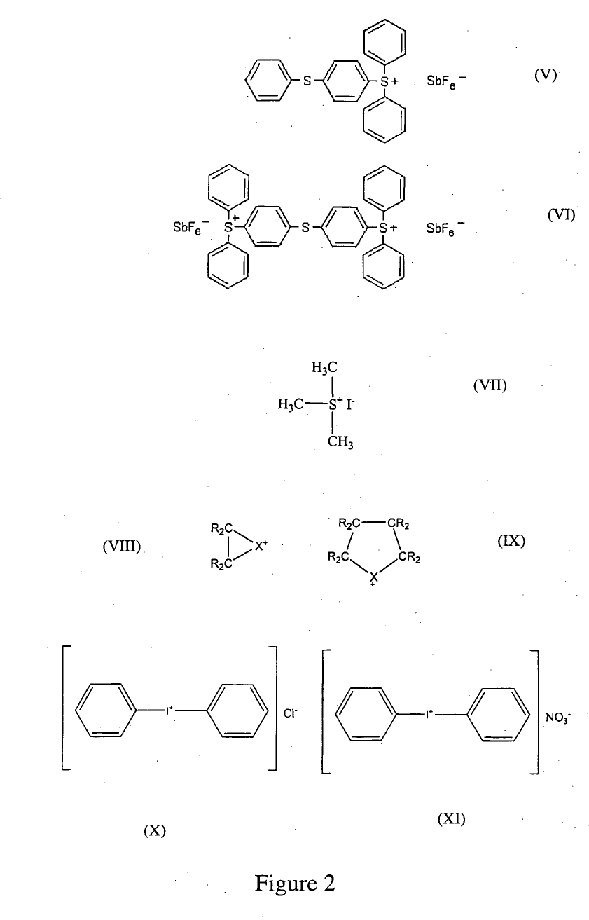

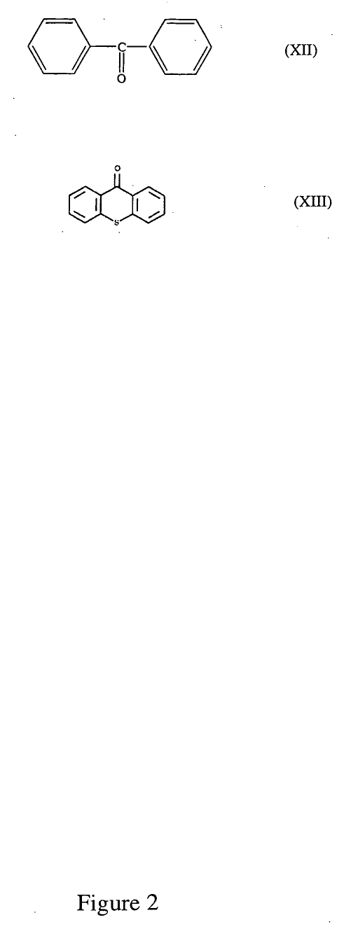

A method for synthesizing polymers within one or more selected region(s) of a solid support is shown in FIGS. 1A-F. In general, the method includes attachment of a first amino acid, linker (or spacer) molecule, or other building block 2 to the surface of a substrate 1. Additionally, mixtures of different building blocks 2 may also be used. For example, in FIG. 1A a first building block 2 can be an amino acid that is attached to a substrate 1 that is comprised of amino-ftinctionalized glass, through the formation of a peptide bond between the carboxylate of the amino acid and the amine group of the glass. The terminal bond-forming site of the building block 2 is protected with a protecting group 3. For example, the α-amino group of an amino acid can be protected with an N-protecting group 3 to prevent unwanted reactivity. If necessary, a side chain of the building block (for example, an R group of an amino acid) may also have a protecting group. Suitable protecting groups include, for example, t-butoxycarbonyl (t-BOC) (FIG. 2, structure (II)), 2-(4-biphenylyl)-2-oxycarbonyl, and fluorenylmethoxycarbonyl (FMOC) (FIG. 2, Structure (III)). Advantageously, embodiments of the present invention are not limited to the type of acid- or base-removable protective group or building block selected.

Referring now to FIG. 1B, once the first polymer building block has been attached to a substrate, a layer of photoresist 4 is deposited over the substrate 1 surface. In embodiments of the invention, the photoresist layer can be created from a solution comprising a polymer, a photosensitizer, and a photo-active compound or molecule in a solvent. The photoresist can be applied using any method known in the art of semiconductor manufacturing for the coating of a wafer with a photoresist layer, such as for example, the spin-coating method. The photoresist-coated substrate is then baked to remove excess solvent from the photoresist for film uniformity.

In FIG. 1C, a photomask 5 is applied over photoresist layer 4. The photomask 5 may be applied using standard techniques and materials used in the semiconductor fabrication industry. For example, the photomask 5 may be a transparent pane, such as a quartz pane, having an emulsion or metal film on a surface creating the mask pattern. Suitable metals include chromium. The pattern of the mask is chosen so that regions on the surface of the substrate can be selectively activated for polymer synthesis. Radiation, for example, ultra violet radiation (UV) or deep ultraviolet radiation (DUV), may then be directed through the photomask 5 onto the photoresist layer. The photoresist 4 is exposed in those regions of the mask that are transparent to the impinging radiation. In general, the device used for creating a pattern in the photoresist can be a physical mask or any other source capable of projecting a pattern image, for example a micromirror.

The exposure of the photoresist 4 to radiation generates cleaving reagents (species that catalyze the removal of a protective group, for example) in the exposed portion of the photoresist layer 4. The generation of cleaving reagents in the photoresist may be the result of a number of processes. For example, the cleaving reagent may result from the direct radiation-induced decomposition of or chemical transformation of a photoactive cleavage reagent precursor compound. Alternatively or in addition, generation of the cleaving reagent may occur through the absorption of light by a photosensitizer followed by reaction of the photosensitizer with the cleavage reagent precursor, energy transfer from the photosensitizer to the cleavage reagent precursor, or a combination of two or more different mechanisms.

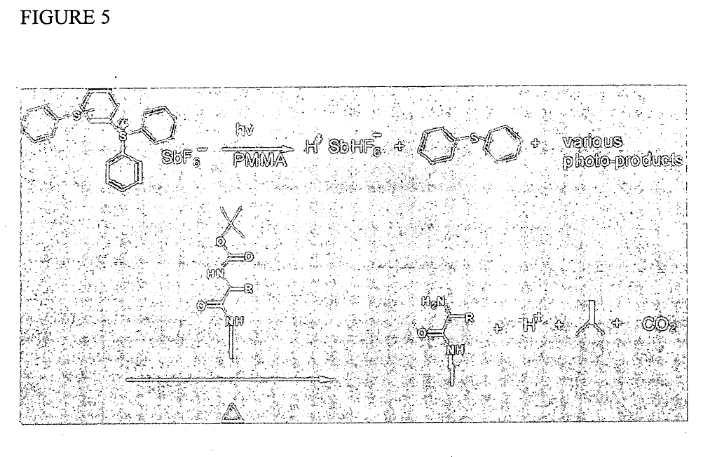

As a result of the radiation-induced generation of the cleaving reagent, the protecting groups 3 are cleaved from the molecules 2 under the exposed area(s) of the photoresist. The molecules 2 located under the unexposed masked regions remain unreacted. The cleaving process leading to the removal of the protecting groups 3 may, for example, be acid-catalyzed cleavage or base-catalyzed cleavage. The chemistry of the process will depend on the type of protecting groups 3 and on the type of cleaving reagents that are generated in the photoresist upon radiation exposure. For example, if the protecting group 3 is t-BOC, acid cleavage can be used. Acids may be generated in the photoresist, for example, through the exposure of sulfonium or halonium salts to radiation (FIG. 2, Structures (IV-VII) and (VIII-IX), respectively). If the protecting group is FMOC, for example, then base cleavage can be used. Cleavage can be accomplished through the reaction of a photogenerated amine or diamine through a decarboxylation process (FIG. 5). The rate of protecting group removal can be accelerated by heating the substrate after the exposure to radiation (post exposure bake). The post exposure bake (PEB) serves multiple purposes in photoresist processing. First, the elevated temperature of the bake drives diffusion of the photoproducts. A small amount of diffusion can be useful in minimizing the effects of standing waves, periodic variations in exposure dose throughout the depth of the film that result from interference of incident and reflected radiation. The other main purpose of the PEB is to drive the acid-catalyzed reaction. Chemical amplification is important because it allows a single photoproduct to cause many solubility-switching reactions, thus increasing the sensitivity of these photoresist systems.

Subsequent to the exposure of the masked substrate to radiation, the photoresist is removed. The photoresist layer 4 may be removed using acetone or another similar suitable solvent. The resulting surface-modified substrate is shown schematically in FIG. 1D. In this structure, there are three regions shown: two regions that have protected molecules and a region having deprotected molecules. The deprotected molecules are available for further reaction, such as for example, a peptide-bond forming coupling reaction whereas the molecules that retain their protective groups are not available for further reaction. Solid phase peptide synthesis can be carried out using standard techniques, see for example, Bodansky, M., Bodansky, A., The Practice of Peptide Synthesis (2^{nd} edition), Springer Verlag, Berlin (1995); Stewart, J. M., Young, J. D., Solid Phase Peptide Synthesis (2^{nd} edition),. Pierce Chemical Company, Rockford Ill., (1984); and Solid-Phase Peptide Synthesis: Methods in Enzymology, vol. 298, Academic Press (1997). FIG. 1E shows a structure resulting from the reaction of the deprotected surface-attached molecules. In FIG. 1E, a building block 6 has been added to molecule 2. Building block 6 may be the same or different from molecule 2. The building block 6 is protected with a protecting group to prevent unwanted reactions.

The processes illustrated in FIGS. 1A-E may be repeated to form polymers on the substrate surface. Through the selection of different mask configurations, different polymers comprising building blocks 2 and 6-10 may be formed in regions upon the surface, as shown schematically in FIG. 2F. In the case where the building blocks are amino acids, peptides having the same or different known sequences are formed in known regions on the surface of the substrate. In general, polymers containing from about 2 to about 50 mers (polymeric units) can be created. In embodiments of the invention peptides having a length of about 6 to about 20 amino acids are created.

Any unreacted deprotected chemical functional groups may be capped at any point during a synthesis reaction to avoid or to prevent further bonding at such molecule. In general, capping reagents can be a reagent that prevents further reactivity at the site of polymer chain formation. Capping groups cap deprotected functional groups by, for example, binding with the unreacted amino functions to form amides. Capping agents suitable for use in an embodiment of the invention include: acetic anhydride, n-acetylimidizole, isopropenyl formate, fluorescamine, 3-nitrophthalic anhydride and 3-sulfoproponic anhydride.

In general, peptides are polymers of amino acids, amino acid mimics or derivatives, and/or unnatural amino acids. The amino acids can be any. amino acids, including α, β, or ω-amino acids. When the amino acids are α-amino acids, either the L-optical isomer or the D-optical isomer may be used. FIG. 2, Structure (I), shows a general structural representation for an amino acid. In general, an amino acid contains an amine group, a carboxylic group, and an R group. The R group can be a group found on a natural amino acid or a group that is similar in size to a natural amino acid R group. Additionally, unnatural amino acids, for example, β-alanine, phenylglycine, homoarginine, aminobutyric acid, aminohexanoic acid, aminoisobutyric acid, butylglycine, citrulline, cyclohexylalanine, diaminopropionic acid, hydroxyproline, norleucine, norvaline, ornithine, penicillamine, pyroglutamic acid, sarcosine,and thienylalanine are also contemplated by the embodiments of the invention. These and other natural and unnatural amino acids are available from, for example, EMD Biosciences, Inc., San Diego, Calif.

A peptide is a polymer in which the monomers are amino acids (natural or unnatural, mimics and derivatives) and which are joined together through amide (peptide) bonds. A peptide can alternatively be referred to as a polypeptide. Peptides contain two or more amino acid monomers, and often more than 50 amino acid monomers (building blocks).

A protein is a long polymer of amino acids linked via peptide bonds and which may be composed of two or more polypeptide chains. More specifically, the term protein refers to a molecule comprised of one or more polymers of amino acids. Proteins are essential for the structure, function, and regulation of the body's cells, tissues, and organs, and each protein has unique functions. Examples of proteins include hormones, enzymes, and antibodies.

A protecting group is a group which is bound to a molecule and designed to block a reactive site in a molecule, but may be removed upon exposure to an activator or a deprotecting reagent. Deprotecting reagents include, for example, acids and bases. Protecting groups can be bound to a monomer, a polymer, a linker molecule or a monomer, or polymer, or a linker molecule attached to a solid support to protect a reactive functionality on the monomer, polymer, or linker molecule. Protective groups that may be used in accordance with an embodiment of the invention include all acid and base labile protecting groups. For example, peptide amine groups are preferably protected by t-butoxycarbonyl (t-BOC or BOC) (shown in FIG. 1, Structure (II)) or benzyloxycarbonyl (CBZ), both of which are acid labile, or by 9-fluorenylmethoxycarbonyl (FMOC) (shown in FIG. 1, Structure (III)), which is base labile.

Additional protecting groups that may be used in accordance with embodiments of the invention include acid labile groups for protecting amino moieties: tert-amyloxycarbonyl, adamantyloxycarbonyl, 1-methylcyclobutyloxycarbonyl, 2-(p-biphenyl)propyl(2)oxycarbonyl, 2-(p-phenylazophenylyl)propyl(2)oxycarbonyl, .alpha. , .alpha.-dimethyl-3,5-dimethyloxybenzyloxy-carbonyl, 2-phenylpropyl(2)oxycarbonyl, 4-methyloxybenzyloxycarbonyl, furfuryloxycarbonyl, triphenylmethyl (trityl), p-toluenesulfenylaminocarbonyl, dimethylphosphinothioyl, diphenylphosphinothioyl, 2-benzoyl-1-methylvinyl, o-nitrophenylsulfenyl, and 1-naphthylidene; as base labile groups-for protecting amino moieties: 9-fluorenylmethyloxycarbonyl, methylsulfonylethyloxycarbonyl, and 5-benzisoazolylmethyleneoxycarbonyl; as groups for protecting amino moieties that are labile when reduced: dithiasuccinoyl, p-toluene sulfonyl, and piperidino-oxycarbonyl; as groups for protecting amino moieties that are labile when oxidized: (ethylthio)carbonyl; as groups for protecting amino moieties that are labile to miscellaneous reagents, the appropriate agent is listed in parenthesis after the group: phthaloyl (hydrazine), trifluoroacetyl (piperidine), and chloroacetyl (2-aminothiophenol); acid labile groups for protecting carboxylic acids: tert-butyl ester; acid labile groups for protecting hydroxyl groups: dimethyltrityl. See also, Greene, T. W., Protective Groups in Organic Synthesis, Wiley-Interscience, NY, (1981).

A linker molecule typically is a molecule inserted into the growing polymer-that does not necessarily convey functionality to the resulting peptide, such as molecular recognition functionality, but instead elongates the distance between the substrate surface and the peptide functionality to enhance the exposure of the peptide functionality on the surface of the substrate. Preferably a linker should be about 4 to about 40 atoms long to provide exposure. The linker molecules may be, for example, aryl acetylene, ethylene glycol oligomers containing 2-10 monomer units (PEGs), diamines, diacids, amino acids, among others, and combinations thereof Examples of diamines include ethylene diamine and diamino propane. Alternatively, the linkers may be the same molecule type as that being synthesized (i.e., nascent polymers), such as polypeptides and polymers of amino acid derivatives such as for example, amino hexanoic acids.

Solid support, support, and substrate refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain embodiments, the solid support may be porous.

Substrate materials useful in embodiments of the present invention include, for example, silicon, bio-compatible polymers such as, for example poly(methyl methacrylate) (PMMA) and polydimethylsiloxane (PDMS), glass, SiO₂ (such as, for example, a thermal oxide silicon wafer such as that used by the semiconductor industry), quartz, silicon nitride, functionalized glass, gold, platinum, and aluminum. Functionalized surfaces include for example, amino-functionalized glass, carboxy functionalized glass, and hydroxy functionalized glass. Additionally, a substrate may optionally be coated with one or more layers to provide a surface for molecular attachment or functionalization, increased or decreased reactivity, binding detection, or other specialized application. Substrate materials and or layer(s) may be porous or non-porous. For example, a substrate may be comprised of porous silicon.

Photoresist formulations useful in the present invention include a polymer, a solvent, and a radiation-activated cleaving reagent. Useful polymers include, for example, poly(methyl methacrylate) (PMMA), poly-(methyl isopropenyl ketone) (PMPIK), poly-(butene-1-sulfone) (PBS), poly-(trifluoroethyl chloroacrylate) (TFECA), copolymer-(α-cyano ethyl acrylate-α-amido ethyl acrylate (COP), and poly-(2-methyl pentene-1-sulfone). Useful solvents include, for example, propylene glycol methyl ether acetate (PGMEA), ethyl lactate, and ethoxyethyl acetate. The solvent used in fabricating the photoresist may be selected depending on the particular polymer, photosensitizer, and photo-active compound that are selected. For example, when the polymer used in the photoresist is PMMA, the photosensitizer is benzophenone, and the photoactive compound is diphenyliodonium chloride, PGMEA or ethyl lactate may be used as the solvent.

In exemplary photoresist formulations, the mass concentration of the polymer may between about 5% and about 50%, the mass concentration of a photosensitizer may be up to about 20%, the mass concentration of the photo-active compound may be between about 1% and 10%, the balance comprising a suitable solvent. After the photoresist is deposited on the substrate, the substrate typically is heated to form the photoresist layer. Any method known in the art of semiconductor fabrication may be used to for depositing the photoresist solution. For example, the spin coating method may be used in which the substrate is spun typically at speeds between about 1,000 and about 5,000 revolutions per minute for about 30 to about 60 seconds. The resulting wet photoresist layer has a thickness ranging between about 0.1 μm to about 2.5 μm.

Catalysts for protective group removal (also referred to as cleaving reagents) useful in the present invention include acids and bases. For example, acids can be generated photochemically from sulfonium salts (FIG. 2, Structures IV-VII), halonium salts (FIG. 2, Structures VIII-IX), and polonium salts (FIG. 2, Structures X-XI). Sulfonium ions are positive ions, R₃S³⁰ , where R is, for example, a hydrogen or alkyl group, such as methyl, phenyl, or other aryl group. Trimethyl sulfdnium iodide and triaryl sulfonium hexafluroantimonatate (TASSbF₆) are shown in FIG. 2, Structures VII and VI, respectively. In general, halonium ions are bivalent halogens, R₂X³⁰ , where R is a hydrogen or alkyl group, such as methyl, phenyl, or other aryl group, and X is a halogen atom. The halonium ion may be linear or cyclic. Polonium salt refers to a halonium salt where the halogen is iodine, the compound R₂I⁺Y⁻, where Y is an amnon, for example, a nitrate, chloride, or bromide. FIG. 2 shows diphenyliodonium chloride and diphenyliodonium nitrate (Structure X and XI, respectively). See also, Frechet, J. M. J., Ito, H., Willson, C. G., Proc. Microcircuit Eng., 260, (1982); Shirai, M., Tsunooka, M., Prog. Polym. Sci., 21:1, (1996); Frechet, J. M. J., Eichler, E, Ito, H., Willson, C. G., Polymer, 24:995, (1983); and Frechet, J. M. J., Ito, H., Willson, C. G., Tessier, T. G., Houlihan, F. M. J., J. of Electrochem. Soc., 133:181 (1986).

Photogenerated bases include amines and diamines having photolabile protecting groups. See for example, Shirai, M., Tsunooka, M., Prog. Polym. Sci., 21:1, (1996); Comeron, J. F., Frechet, J. M. J., J. Org. Chem., 55:5919, (1990); and Comeron, J. F., Frechet, J. M. J., J. Am. Chem. Soc., 113:4303, (1991).

Optionally, the photoresists useful in the present invention may also include a photosensistizer. In general, a photosensitizer absorbs light and interacts with the cleavage reagent precursor, through one or more mechanisms, including, reaction with the cleavage reagent precursor and energy transfer from the photosensitizer to the cleavage reagent precursor. Useful photosensitizers include, for example, benzophenone (FIG. 2, Structure XII) and other similar diphenyl ketones, thioxanthenone (FIG. 2, Structure XIII), anthraquinone, fluorenone, acetophenone, and perylene.

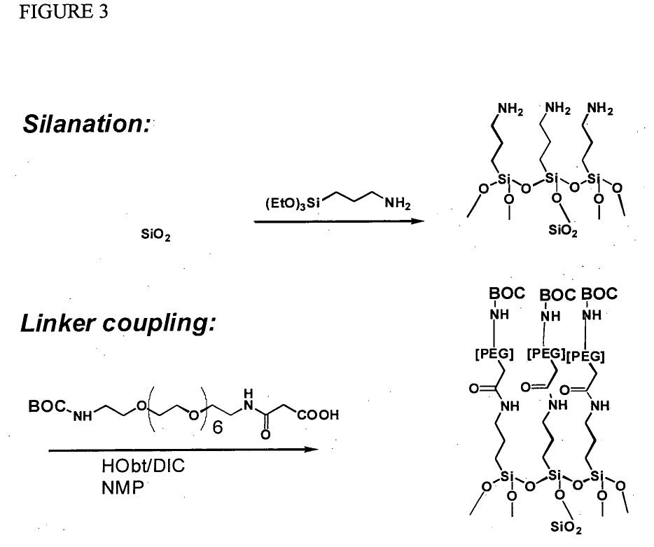

FIG. 3 provides a method for derivatization of a SiO₂ surface and linking of polymeric molecules to the surface. In FIG. 3 the SiO₂ surface is silanated by reacting it with aminopropyltriethoxy silane (APTES). The resulting surface presents an amine functional group for further reaction, such as peptide bond formation. Modulation of the density of polymers on the surface can be attained by silanation. For example, density can be modulated by mixing a functionalizable silane for example, APTES, with a non-functional silane (a silane with no non silyl functional group), for example, propyltrialkoxy silane. The derivatized surface can then be reacted with a linker. In this example, the linker is a polyethylene glycol molecule having an amine group protected with BOC at one terminus and a peptide-bond forming group at the second terminus. This coupling reaction can be accomplished in a solution of 1-hydroxybenzotriazole (HOBt) and diisopropylcarbodiimide (DIC) in N-methyl pyrrolidone (NMP). The linker molecule serves to separate polymer (peptide) that is subsequently synthesized from surface of the substrate.

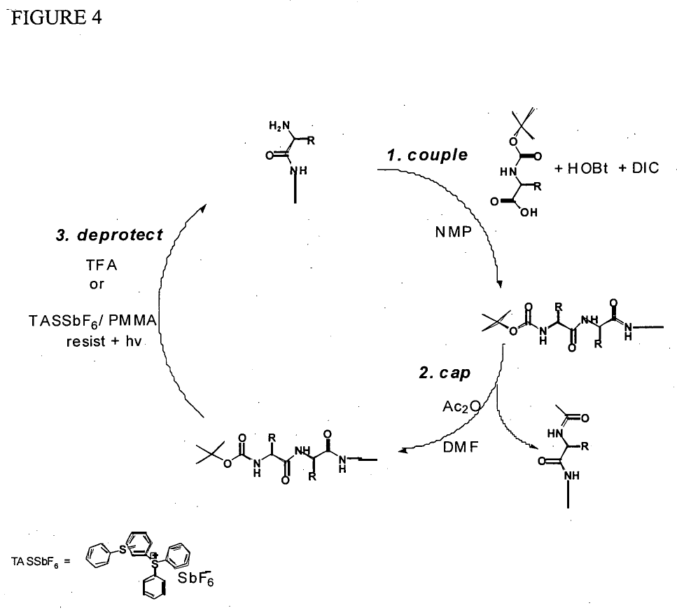

FIG. 4 shows a general scheme for solid-phase peptide synthesis. A substrate surface is provided having a first amino acid attached to the surface. A second amino acid having a protecting group is coupled to the first amino acid. In this example, the second amino acid is N-protected with a BOC protecting group. The coupling reaction is performed in a solution of 1-hydroxybenzotriazole (HOBt) and diisopropylcarbodiimide (DIC) in N-methyl pyrrolidone (NMP). Unreacted amine groups are capped using an acetic anhydride (Ac₂O) solution in dimethylformamide (DMF). The substrate surface is then coated with a photoresist. In this example, the photoresist is comprised of PMMA polymer, TASSbF₆ (photoactivated acid generator), and PGMEA (as a solvent). (In FIG. 4, TFA represents trifluoroacetic acid, the acidic cleaving reagent typically used for bulk solid-phase peptide synthesis. Experiments demonstrated that yields for a peptide synthesis process according to the current invention were similar to yields for bulk solid-phase synthesis procedures using TFA as a protecting group removal catalyst.) Upon exposure to radiation, in this case UV radiation, an acid is produced in the photoresist and the N-protecting group is removed from the attached peptide in the region of UV exposure. By repeating the process shown in FIG. 4, peptides of desired sequence and length is selected regions upon the substrate surface can be produced.

FIG. 5 shows the photogeneration of an acid and the deprotection of an amine group of a surface-attached amino acid. In this example, the phoactivated acid generator is triaryl sulfonium hexafluroantimonatate (TASSbF₆), and the photoresist is comprised of PMMA polymer. Upon exposure to light, an acid, H³⁰ SBHF₆⁻, is generated. The rate of the resulting deprotection of the surface-attached reactive group, in this case an amine, is enhanced through heating the substrate. In the case of a t-BOC protecting group, the deprotection reaction liberates a surface-attached amine, a proton, an acetone molecule, and a carbon dioxide molecule.

### EXAMPLE 1

A glass substrate was silanated using a solution of 3% APTES (aminopropyl triethoxy silane) in 95% ethanol. The surface of the substrate was then washed and annealed at about 100° C. for about 1 hour. The substrate was then treated with a 1:1 solution of DIEA (diisopropyl ethyl amine) in DMF (dimethylformamide). A spacer molecule was then coupled to the surface using a solution of 0.25 M solution of O-(N-Boc-2-aminoethyl)-O′-(N-diglycolyl-2-aminoethyl) hexaethyleneglycol, 0.25 M HOBt, and 0.25 M DIC (diisopropylcarbodiimide) in NMP (N-methyl pyrrolidone)and gentle agitation over the surface of the substrate in a sealed container for about 30 min. The solution was then discarded and the surface replenished with fresh solution. After coupling was complete, the surface was washed with NMP and then acetone. Unreacted surface amine groups were capped by treatment with 1:1 acetic anhydride in DMF solution (a 50% acetic anhydride solution in DMF) for about 30 minutes. The surface was then washed.

A photoresist was prepared by mixing about 10% by mass of PMMA, 20% by mass of triarylsulfonium hexafluoroantimonate in PGMEA solvent and spin coating the mixture over the amino acid derivatized glass surface for about 60 seconds at 2,000 rpm. The photoresist may also optionally contain thioxanthenone, a photosensitizer. The photoresist layer was baked at about 85° C. for about 90 seconds. The resulting photoresist layer had a thickness of about 2 μm.

Acid was generated in the photoresist layer by irradiation of the surface of the substrate with 2-3 J of 365 nm UV light through a mask. The reaction was accelerated by a post exposure bake at about 65° C. for about 60 seconds. After the photogenerated acid deprotection was achieved, the surface of the substrate was rinsed with acetone to strip the photoresist from the surface and the surface was dried. The surface was neutralized by treatment in 25% DIEA/DMF for about 5-10 minutes and then washed in DMF.

A second amino acid (Boc-Leu-OH) was coupled to the surface of the substrate using a 0.25 M solution of N-α-Boc-Leu, HOBt, and DIC as above. Subsequent rounds of coupling and deprotection were accomplished by repeating the above procedures to generate peptides of a desired length. As a result, a hexamer peptide, SDLYKL segement of human tumor suppressor protein p53, was synthesized on an APTES surface derivatized with a PEG (polyethylene glycol) spacer. A labeled F1-tagged anti-p53 monoclonal antibody in a standard Ab binding assay recognized and strongly bound to the SDLYLK peptide on the surface as determined by fluorescence detection.

### EXAMPLE 2

An array of wildtype (SDLHKL) and mutant (AGLHKL) peptide was synthesized on an aminated glass surface with a linker molecule, O-(N-Boc-2-aminoethyl)-O′-(N-diglycolyl-2-aminoethyl) hexaethyleneglycol, for spacing the peptides from the surface. The peptides were synthesized in a checkerboard pattern using uniform photodeprotection of t-Boc protecting groups through an open grid mask till the second leucine and spatially localized deprotection through a checkerboard mask for the last two amino acid couplings.

The photodeprotection and coupling of linker molecules and amino acids was carried out as described in Example 1.

The peptide array was incubated for 1 hour with 5 μg/ml monoclonal antibody known to specifically recognize the SDLHKL epitope of human p53 protein. A second incubation was performed with fluorescein-labeled rabbit antibody raised against mouse antibody at a 1:100 dilution in phosphate buffered saline with 0.05% Tween 20. A fluorescent checker board pattern was detected on fluorescence scanming of the array suggesting specific interaction of antibody with the wildtype sequence.

### EXAMPLE 3

Photoresist formulations may include a sensitizer in addition to the photogenerated acid catalyst to generate the acid deprotection catalysts. In general, the amount of PMMA in the resist in these exemplary formulations may vary between about 3% and about 50%.

Useful photoresists may be made using diaryliodonium salts (DAI) and photosensitizers. The mass ration between DAI and photosensitizer may be between about 1:10 and 1:1. For instance, (tolylcumyl)polonium tetrakis (pentafluorophenyl) borate with isopropyl-9H-thioxanthen-9-one may be formulated in a 1:10 or 1:1 (or a ratio there between) in PMMA and PGMEA to form final concentrations of between about 0.5% to 10% by mass DAI. The formulation selected may be spun coated on the substrate surface and baked. The radiation exposure dose may be between about 0.02 J and about 10 J. Post exposure baking may be conducted for about 30 to 60 seconds at about 40° C. to about 85° C.

1. A method for making an array of polymers comprising,
attaching to a substrate surface a first molecule capable of forming a peptide bond wherein the molecule contains a protecting group that prevents the formation of a peptide bond,
depositing a photosensitive layer over the substrate surface wherein the photosensitive layer contains a photo-active compound that upon activation generates a second compound capable of causing the removal of the protecting group,
exposing a portion of the substrate surface to ultraviolet radiation wherein ultraviolet radiation exposure causes the removal of protecting groups, removing the photosensitive layer, and
coupling a second molecule capable of forming a peptide bond, wherein the molecule contains a protecting group that prevents the formation of a peptide bond, to the first molecule capable of forming a peptide bond that has been deprotected.

2. The method according to claim 1 also including baking the substrate after exposing a portion of the substrate surface to ultraviolet radiation.

3. The method according to claim 1 also including capping unreacted peptide bond-forming sites on the first molecule capable of forming a peptide bond after coupling the second molecule capable of forming a peptide bond.

4. The method according to claim 1 wherein attaching is accomplished through the formation of a peptide bond.

5. The method according to claim 1 wherein the second compound capable of causing the removal of the protecting group is a photogenerated acid or base.

6. The method according to claim 1 wherein the second compound capable of causing the removal of the protecting group is a photogenerated acid and the photo-active compound is selected from the group consisting of sulfonium salts, halonium salts, and polonium salts.

7. The method according to claim 1 wherein the substrate surface to which the first molecule capable of forming a peptide bond is attached is an amino-functionalized SiO₂ surface.

8. The method according to claim 7 wherein the substrate is comprised of silicon having a layer of SiO₂ on the surface.

9. The method according to claim 1 wherein the photosensitive layer comprises a polymer, a photo-active compound, and a solvent.

10. The method according to claim 1 wherein the photosensitive layer additionally includes a photosensitizer.

11. The method according to claim 10 wherein the photosensitizer is selected from the group consisting of benzophenones, thioxanthenones, anthraquinone, fluorenone, acetophenone, and perylene.

12. The method of claim 1 wherein one or more of the molecules capable of forming a peptide bond are selected from the group consisting of natural and unnatural amino acids.

13. The method according to claim 1 wherein the elements of depositing a photosensitive layer, exposing a portion of the substrate surface, removing the photosensitive layer, and coupling a second molecule are repeated a plurality of times.

14. The method according to claim 13, wherein a resulting peptide attached to the substrate surface has a length from about 3 peptide bonds to about 25 peptide bonds.

15. The method of claim 1 wherein a molecule capable of forming a peptide bond is a spacer molecule selected from the group consisting of aryl acetylenes, polyethyleneglycols, nascent polypeptides, diamines, and diacids.

16. The method of claim 1 wherein a feature size of the array is less than 100 μm².

17. The method of claim 1 wherein the array contains 1,000 to 10,000 features.

18. The method of claim 1 wherein the protecting group is t-butoxycarbonyl, benzyloxycarbonyl, or 9-fluorenylmethoxycarbonyl.

19. A method for making an array of polymers comprising,
modulating the density of peptides to be formed on a substrate surface by blocking a fraction of the possible attachment sites from molecular coupling, attaching to the substrate surface a first molecule capable of forming a peptide bond wherein the molecule contains a protecting group that prevents the formation of a peptide bond,
depositing a photosensitive layer over the substrate surface wherein the photosensitive layer contains a photo-active compound that upon activation generates a second compound capable of causing the removal of the protecting group,
exposing a portion of the substrate surface to ultraviolet radiation wherein ultraviolet radiation exposure causes the removal of protecting groups, removing the photosensitive layer, and
coupling a second molecule capable of forming a peptide bond, wherein the molecule contains a protecting group that prevents the formation of a peptide bond, to the first molecule capable of forming a peptide bond that has been deprotected.

20. The method of claim 19 also including baking the substrate after exposing a portion of the substrate surface to ultraviolet radiation.

21. The method of claim 19 wherein modulating the density of peptides to be formed on the substrate is accomplished by coupling a mixture of molecules capable of forming a peptide bond to the substrate surface wherein the mixture contains molecules having a protecting group that prevents the formation of a peptide bond and molecules having a capping group that prevents the formation of a peptide bond.

22. The method according to claim 19 also including capping unreacted peptide bond-forming sites on the first molecule capable of forming a peptide bond after coupling the second molecule capable of forming a peptide bond.

23. The method according to claim 19 wherein attaching is accomplished through the formation of a peptide bond.

24. The method according to claim 19 wherein the second compound capable of causing the removal of the protecting group is a photogenerated acid or base.

25. The method according to claim 19 wherein the second compound capable of causing the removal of the protecting group is a photogenerated acid and the photo-active compound is selected from the group consisting of sulfonium salts, halonium salts, and polonium salts.

26. The method according to claim 19 wherein the substrate surface to which the first molecule capable of forming a peptide bond is attached is amino-functionalized SiO₂ surface.

27. The method according to claim 19 wherein the substrate is comprised of silicon having a layer of SiO₂ on the surface.

28. The method according to claim 19 wherein the photosensitive layer comprises a polymer, a photo-active compound, and a solvent.

29. The method according to claim 19 wherein the photosensitive layer additionally includes a photosensitizer.

30. The method according to claim 29 wherein the photosensitizer is selected from the group consisting of benzophenones, thioxanthenones, anthraquinone, fluorenone, acetophenone, and perylene.

31. The method of claim 19 wherein one or more of the molecules capable of forming a peptide bond are selected from the group consisting of natural and unnatural amino acids.

32. The method according to claim 19 wherein the elements of depositing a photosensitive layer, exposing a portion of the substrate surface, removing the photosensitive layer, and coupling a second molecule are repeated a plurality of times.

33. The method according to claim 32, wherein a resulting peptide attached to the substrate surface has a length from about 3 peptide bonds to about 25 peptide bonds.

34. The method of claim 19 wherein a molecule capable of forming a peptide bond is a spacer molecule selected from the group consisting of aryl acetylenes, polyethyleneglycols, nascent polypeptides, diamines, and diacids.

35. The method of claim 19 wherein a feature size of the array is less than 100 μm².

36. The method of claim 19 wherein the array contains 1,000 to 10,000 features.

37. The method of claim 19 wherein the protecting group is t-butoxycarbonyl, benzyloxycarbonyl, or 9-fluorenylmethoxycarbonyl.
